SEQUENCE LISTING (11 sequences)

SEQ ID NO: 1 - DNA, 21 bp, Artificial Sequence
  Sequence of the NRSE motif.
ttcagcaccacggagagtgcc

SEQ ID NO: 2 - DNA, 21 bp, Artificial Sequence
  Consensus Sequence of the NRSE motif.
ttcagcaccacggacagcgcc

SEQ ID NO: 3 - DNA, 21 bp, Artificial Sequence
  General Sequence of the NRSE motif.
nncagcaccnngganagnnnc

SEQ ID NO: 4 - DNA, 21 bp, Artificial Sequence
  NRSE motif.
ttcagcaccacggagagtgcc

SEQ ID NO: 5 - DNA, 21 bp, Artificial Sequence
  NRSE motif.
ttcagcaccgcggacagtgcc

SEQ ID NO: 6 - DNA, 21 bp, Artificial Sequence
  NRSE motif.
ttcagcacctcggacagcatc

SEQ ID NO: 7 - DNA, 21 bp, Artificial Sequence
  NRSE motif.
ttcagcaccgcggagagcgtc

SEQ ID NO: 8 - DNA, 21 bp, Artificial Sequence
  NRSE motif.
tccagcaccgtggacagagcc

SEQ ID NO: 9 - DNA, 21 bp, Artificial Sequence
  NRSE motif.
ttcagcaccgaggacggcgga

SEQ ID NO: 10 - DNA, 21 bp, Artificial Sequence
  NRSE motif.
atcagcaccacggacagcggc

SEQ ID NO: 11 - DNA, 21 bp, Artificial Sequence
  NRSE motif.
ttcagcacctaggacagaggc

(19) United States
(12) Patent Application Publication
(10) Pub. No.: US 2006/0121013 A1
(43) Pub. Date: Jun. 8, 2006

(54) Use of negative regulatory elements for the neurospecific expression of transgenes

(75) Inventors: Jacques Mallet, Paris (FR); Helene Kiefer, Bitches (FR); Stephanie Millecamps, Roncg (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Centelion, Vitry sur Seine (FR)

(21) Appl. No.: 11/256,112

(22) Filed: Oct. 24, 2005

Related U.S. Application Data
(62) Division of application No. 09/601,898, filed on Sep. 15, 2000, now abandoned.

(30) Foreign Application Priority Data
Feb. 12, 1998 (FR) FR98-01715

(51) Int. Cl. A61K 48/00; C12N 5/08; C12N 15/867

(52) U.S. Cl. 424/93.21; 514/44; 435/368; 435/456

(57) ABSTRACT

The invention concerns novel methods and constructs for controlling nucleic acid expression, in particular methods and constructs using NRSE sequences for obtaining a targeted expression of transgenes in nerve cells.

The present invention relates to novel methods, constructs and vectors containing these constructs which make it possible to regulate the expression of a nucleic acid. In particular, the invention relates to methods, constructs and vectors which make it possible to obtain targeted expression of transgenes in nerve cells in vivo or ex vivo. The invention is particularly suited to applications in which genes are transferred in vivo, for example for therapeutic or scientific approaches.

In vivo or ex vivo gene therapy permits the local and efficient transfer of genes encoding factors of interest (transgenes), in particular into the nervous system of a host organism. In this regard, various types of vector have been successfully employed for transferring different types of transgene into nerve cells in vivo or ex vivo. Vectors which may be cited are, in particular, viral vectors of the adenovirus, AAV or HSV type, or certain non-viral vectors of the cationic polymer type (polyethyleneimine, for example). Thus, these vectors have made it possible to transfer transgenes stably and efficiently into cells of the nervous system in vivo (WO 94/08026, WO 93/09239 and WO 96/02655). The possibility of being able to carry out this type of transfer affords a large number of applications, in particular in the medical field. Thus, these vectors can be used in gene therapy approaches in vivo or ex vivo. In this regard, various preclinical studies relating to the transfer of trophic factors into the nervous system are currently in progress. These vectors can also be used for creating transgenic animals, enabling compounds to be tested, or else for different labelling or bioavailability studies.

In all these applications, even if highly efficient and stable transfer appears to have been achieved, it is essential to be able to regulate expression of the transgenes. In this respect, various systems have been described for attempting to restrict expression of transgenes solely to particular tissues, for example by using so-called “tissue-specific” promoters or complex chimeric systems which require the use of several constructs and/or regulatory factors. As an example of a tissue-specific promoter, the glial fibrillary acidic protein promoter, which is mainly active in glial nerve cells, may in particular be mentioned. However, the regulatory systems which are currently available or validated suffer from certain drawbacks, in particular from the fact that their strength and/or selectivity is not always satisfactory. Thus, in the large majority of known systems, selectivity is generally obtained to the detriment of the strength of the promoter and it is therefore difficult to achieve expression which is at once substantial, stable and specific.

The present invention provides methods, constructs and vectors containing these constructs which make it possible to remedy the drawbacks of the prior art. In particular, the present invention describes a chimeric promoter which enables expression to be targeted neuronally. In this regard, the invention makes use of negative regulatory elements which prevent the genes from being expressed in non-neuronal cells. This type of construct exhibits several advantages as compared with the previous systems. On the one hand, since repressor sequences and ubiquitous promoters are often short and well-characterized, they can be readily combined with other regulatory elements. On the other hand, these regulatory elements can in theory be combined with any ubiquitous promoter and therefore represent an astute system which enables neurospecific expression of transgenes of interest to be obtained in a simple manner, even when the expression vector employed has little or no tropism for nerve cells.

A large number of studies describe cis sequences which repress transcription of neuronal genes in non-neuronal cells. One particular type of sequence which is endowed with this property consists of NRSEs (“neurone restrictive silencer elements”) also termed RE-1s (repressor element-1). NRSE is a short sequence, of approximately 21 bp in size, which has been demonstrated to be present upstream of several neuronal genes: for example SCG10 [1], sodium II channel [2] and synapsin I [3]. Furthermore, analogous sequences have been found upstream of a large number of other neuronal genes, even if their functionality has still not been demonstrated [4]. These NRSE sequences repress the non-neuronal expression of genes by binding a specific transcription factor, i.e. the NRSF (neurone restrictive silencer factor) protein, also termed REST (RE-1 silencing transcription factor), which is exclusively present in non-neuronal cells [5]. This protein belongs to the family of zinc finger transcription factors. It intervenes during development in order to initiate the repression of neuronal genes in non-neuronal cells, and is then involved in adulthood in the maintenance of this repression.

Research groups which have characterized the presence of NRSE sequences upstream of neuronal genes have verified the ability of these sequences to decrease the expression of reporter genes following transfection of non-neuronal cells. In order to do this, they have combined their NRSE sequence with a minimal heterologous promoter, that is a promoter which only allows basal transcription. Thus, when placed upstream of the thymidine kinase promoter, for example, a copy of the calcium II channel NRSE sequence reduces expression of the reporter gene by half in non-nerve cells [9]. Similarly, two copies of the synapsin I NRSE sequence combined with the minimal c-fos promoter reduce expression in non-nerve cells by 75% [3]. Furthermore, the presence of an NRSE sequence also enabled the activity of the SV40 promoter to be regulated in non-neuronal cells [10].

However, the NRSE sequences have never been used for directing the expression of a strong ubiquitous promoter, nor from a viewpoint of gene therapy. Thus, it has never been shown whether such sequences are able to suppress the expression which is induced by a strong promoter. Similarly, it has never been shown whether these sequences are still active in vivo when placed in a chimeric environment. More generally, it has never so far been demonstrated that these sequences are active in vivo when combined with heterologous promoters.

The invention relates to recombinant nucleic acids which permit the neurospecific expression of genes. It also relates to vectors which include these nucleic acids, in particular of viral origin, as well as to cells which contain these nucleic acids and/or vectors. The invention also relates to specific chimeric promoters which are suitable for expressing genes neurospecifically in the nervous system in vivo. The invention also relates to compositions which comprise these elements and to their use for transferring genes.

A first aspect of the invention therefore lies in a recombinant nucleic acid which comprises:
a promoter
one or more NRSE sequences, and
a therapeutic gene.

The constructs according to the invention therefore comprise a region for repressing, in non-neuronal cells, the active expression of the therapeutic gene which is induced by the promoter. This repressor region consists of one or more NRSE motifs.

The NRSE motif which is employed within the context of the present invention advantageously comprises all or part of the following 21 bp sequence: TTCAGCACCACGGAGAGTGCC (SEQ ID No. 1)

This sequence corresponds to the NRSE sequence of the SCG10 gene [2]. Nevertheless, it is to be understood that the NRSE motif which is employed within the context of the invention can include some variations as compared with this sequence, in so far as it retains the abovementioned repressor properties. Thus, sequences of the NRSE type which exhibit some degree of variation have been observed in various genes, as described in the article by Schoenherr et al. [4], which is hereby incorporated into the present application by reference. Based on the variants which have been observed, an NRSE consensus sequence, which corresponds to the sequence which is most frequently encountered, has been defined. This sequence is: TTCAGCACCACGGACAGCGCC (SEQ ID No. 2). Preferred variants within the meaning of the invention comprise substitutions involving 1 to 5 base pairs of this SEQ ID No. 2 sequence. More preferably, they comprise variations involving from 1 to 3 base pairs. In this regard, the variations preferably involve residues 1, 2, 10, 11, 15, 18, 19 and/or 20 of the above sequence. The substitutions can correspond to the deletion or replacement of the base concerned with any other base. Thus, an NRSE motif can be represented more generally by all or part of the following sequence: NNCAGCACCNNGGANAGNNNC (SEQ ID No. 3) in which N denotes a base selected from A, T, C and G. The following are, in particular, specific examples of NRSE motifs which can be used within the context of the present invention:

| | |
|---|---|
| TTCAGCACCACGGACAGCGCC | (SEQ ID No. 2) |
| TTCAGCACCACGGAGAGTGCC | (SEQ ID No. 4) |
| TTCAGCACCGCGGACAGTGCC | (SEQ ID No. 5) |
| TTCAGCACCTCGGACAGCATC | (SEQ ID No. 6) |
| TTCAGCACCGCGGAGAGCGTC | (SEQ ID No. 7) |
| TCCAGCACCGTGGACAGAGCC | (SEQ ID No. 8) |
| TTCAGCACCGAGGACGGCGGA | (SEQ ID No. 9) |
| ATCAGCACCACGGACAGCGGC | (SEQ ID No. 10) |
| TTCAGCACCTAGGACAGAGGC | (SEQ ID No. 11) |

In addition, these motifs can also include, at one or other end or at both ends, additional bases which do not interfere with the abovementioned repressor activity. In particular, these additional bases can comprise restriction sites, neutral sequences or sequences which are derived from cloning steps and which contain, for example, regions of a plasmid or of a vector or sequences which flank the NRSE motif in the gene of origin.

These different motifs may be prepared by any technique known to the skilled person for preparing nucleic acids. Thus, they may be prepared by the techniques of automated nucleic acid synthesis using commercial synthesizers. They may also be obtained by screening DNA libraries, for example by hybridizing with specific probes or by carrying out experiments involving binding to an NRSF factor. In addition, any other variant of the SEQ ID No. 1 sequence can be identified in DNA libraries by means of homology searching, for example.

Once synthesized, the NRSE-type motif can then be tested functionally. For this, a first test consists, in particular, in determining the ability of the motif to bind the NRSF factor. This can be carried out under various conditions, for example by performing gel migration retardation experiments in accordance with the technique described by Mori et al. [2] or by Schoenherr et al. [5], which publications are incorporated into the present publication by reference. Subsequently, the ability of the motif to repress expression can be determined by inserting this motif into a test plasmid which contains a reporter gene (chloramphenicol acetyltransferase, luciferase, lacZ, etc.) under the control of a promoter and comparing the levels of expression of the said reporter gene in nerve cells and in non-nerve cells. This type of methodology is described, for example, in Schoenherr et al. [4, 5] and in the examples. Preferably, the motif is regarded as being functional when it generates an expression differential between the nerve cells and the non-nerve cells of at least 40%. More preferably, this differential is at least 50% and, advantageously, at least 60%.

As indicated previously, the nucleic acids of the invention can comprise one or more NRSE motifs as defined above. When several motifs are present, the same motif may be repeated several times or several variants may be present. Preferably, the constructs of the invention comprise the same motif which is repeated several times. The constructs can comprise up to 50 motifs. Advantageously, the motif is repeated from 2 to 20 times, preferably from 3 to 15 times. As illustrated in the examples, favourable results have been obtained with the motif being repeated 3, 6 or 12 times, with particularly significant results being obtained with 6 and 12 repeats.

The NRSE motifs can be inserted into the constructs of the invention in any untranscribed or untranslated region or in an intron. Advantageously, they are placed in the 5′ non-coding regions and even more preferably in the proximal region of the promoter. Furthermore, since the activity of these motifs is independent of their orientation, they can be inserted either in the transcription sense or in the opposite orientation. Finally, when several motifs are used, they are preferably inserted in tandem, in one and the same region of the construct. However, it is to be understood that they can still be inserted in different regions.

The second element which enters into the composition of the nucleic acids of the invention is a promoter which enables the transgene to be expressed in the intended cell. Advantageously, the promoter is a promoter which is active in nerve cells or tissues, and in particular a eukaryotic promoter. In this regard, the promoter can, for example, be a ubiquitous promoter, that is a promoter which functions in most cell types. Still more preferably, the promoter can, therefore, be a ubiquitous eukaryotic promoter. The promoter can be autologous, that is a promoter which derives from the same species as the cell in which expression is sought, or a xenogenic promoter (derived from another species). Advantageous examples of ubiquitous eukaryotic promoters which may be mentioned are strong promoters, such as the promoter of the phosphoglycerate kinase 1 (PGK) gene. A so-called strong promoter is understood as being any promoter whose activity is comparable to that of the viral promoters. In eukaryotes, PGK is an enzyme which is involved in glycolysis. In mice, the promoter of this gene, which is approximately 500 bp in size, comprises a so-called enhancer region (−440/−120) and a promoter region (−120/+80) which contains several transcription initiation sites [6]. The efficacy of this PGK promoter has already been demonstrated in previous experiments on gene transfer in vitro and in vivo [7, 8].

According to one preferred embodiment, the invention relates to a nucleic acid which comprises the PGK promoter and one or more NRSE sequences. As illustrated in the examples, this type of construct makes it possible to direct the neurospecific expression of a transgene. In this regard, the invention also relates to a chimeric promoter which comprises a strong ubiquitous promoter and one or more NRSE sequences. Thus, the invention demonstrates that these NRSE sequences can be used to repress the activity of a strong promoter efficiently, including in vivo. This therefore makes it possible to use these novel promoters in a large number of applications. A specific chimeric promoter within the meaning of the invention is shown by the designation xNRSE-PGK, in which x is an integer from 1 to 50, preferably from 1 to 20.

Examples of other ubiquitous eukaryotic promoters which can be used within the context of the present invention are the promoters which direct the expression of the genes of the obligatory cell metabolism (these genes are termed “domestic” genes or “housekeeping” genes and specify proteins which are required for functions which are common to all cells). Examples of these genes are genes which are involved in the Krebs cycle, in cellular respiration or in the replication or transcription of other genes. Specific examples of this type of promoter which may be mentioned are the promoters of the α1-antitrypsin, β-actin, vimentin, aldolase A or Ef1α (elongation factor) genes.

The promoter which is employed within the context of the invention can also be a eukaryotic promoter of neurospecific type, thereby improving its neurospecificity. Examples of these promoters which may be mentioned are the promoters of the NSE (neurone-specific enolase), NF (neurofilament), TH (tyrosine hydroxylase), DAT (dopamine transporter), CHAT (choline acetyl transferase), DBH (dopamine β-hydroxylase), TPH (tryptophan hydroxylase) and GAD (glutamic acid dehydrogenase) genes and, more generally, all the promoters of enzymes for synthesizing neuromediators or of transporters of neuromediators, or any other promoters of genes whose expression is specific for a given neuronal or glial type or subtype.

Finally, it is also possible to envisage using viral promoters, for example CMV (cytomegalovirus), RSV (Rous sarcoma virus), TK (thymidine kinase), SV40 (simian virus) and LTR (long terminal repeat) promoters.

Furthermore, the nucleic acids of the invention also comprise a therapeutic gene. Within the meaning of the present invention, a therapeutic gene is understood as being any nucleic acid which comprises at least one open reading frame which encodes an RNA or a therapeutic or vaccinating polypeptide. The nucleic acid can be a complementary, genomic, synthetic or semisynthetic DNA. It can have a variety of origins, such as mammalian, plant, viral, artificial, etc. The transcription or translation product therefore exhibits therapeutic or vaccinating properties. Specific examples which may be mentioned are enzymes, growth factors (in particular trophic factors), neurotransmitters or their precursors, toxic factors (thymidine kinase, for example), antibodies or antibody fragments, etc.

It is possible to envisage using the constructs of the invention for directing the expression of one or more genes encoding an RNA or a protein which is to be intended for neurones without this gene or these genes being expressed in non-neuronal cells, in order to establish animal models or from the viewpoint of a substitutive or suppressive, etiological or symptomatic therapy. The genes may, for example, be genes of the trophic factor family, such as, for example, neurotrophins (NGF, BDNF, NT3, NT4-5, GMF, etc.), growth factors [fibroblast growth factor (FGF) (a and b) family, vascular endothelial cell growth factor (VEGF) family, epidermal growth factor (EGF) family and insulin growth factor (IGF) (I and II) family], and the TGFβ superfamily, including the TGFβ and GDNF/neurturin families.

The genes can also be genes which encode cytokines, for example CNTF, LIF, oncostatin M or cardiotrophin 1, or proteins of the interleukin family.

The genes can also be genes which encode the receptors for these different factors or which encode transcription factors which regulate the expression of these different factors. The genes can also be genes which encode the enzymes for synthesizing or degrading the different neurotransmitters and neuropeptides or their precursors, or cofactors which are essential to this synthesis or this catabolism, or else transcription factors which regulate the expression of these proteins; the genes can also be genes which encode the receptors for the neurotransmitters/neuropeptides (or encode sub-units of these receptors) and encode the proteins which are involved in the transduction pathways.

Other genes of interest within the context of the invention are, in particular, genes which encode antioxidants, for example SOD (superoxide dismutase), GPX (glutathione peroxidase), catalase or an enzyme of cell respiration; the enzymes involved in the cell cycle, such as p21, or other protein inhibitors of dependant kinases, and also the genes of apoptosis, such as ICE, Bcl2, BAX, etc.

More generally, any gene whose anomalous expression induces a pathology of the nervous system may be expressed in the constructs of the invention, such as genes whose mutation is directly at the origin of pathologies or genes whose products are involved in the same metabolic pathway. The genes may also be toxic genes for antineoplastic therapy (for example thymidine kinase or cytosine deaminase), genes for antisense RNAs or ribozymes, or else reporter genes for developmental, kinetic and/or bioavailability studies, such as the β-galactosidase, luciferase or GFP (green fluorescent protein) genes. The genes may also be genes which are involved in conditional recombination systems in the nervous system, in order to establish animal models, such as transgenic animals, including the conditional knockout system.

It is understood that the skilled person is easily able to suit the type of gene to the sought-after use.

In the nucleic acids of the invention, the different elements are arranged such that the promoter controls the expression of the therapeutic gene, and the NRSE sequence(s) control(s) the activity of the promoter. In general, the gene is therefore placed downstream of the promoter and in phase with it. Furthermore, the regulatory region is generally placed upstream of the promoter although, as indicated previously, this is not a prerequisite for activity. The distance between the regulatory region (NRSE sequences) and the promoter varies in accordance with the nature of the sequences employed and the number of repeats of the NRSE motif. Advantageously, the regulatory sequences are placed at a distance of less than 2 kb from the promoter, preferably at a distance of less than 1 kb.

According to one particular embodiment of the invention, the NRSE sequences are combined with a regulatory system in order finely to control the expression of nucleic acids in the cells. The regulatory system which may quite particularly be mentioned is the system which on the one hand uses the tetracycline-controlled transactivator tTA and on the other hand uses a tTA-sensitive promoter such as that described in application FR 98/140080, which is hereby incorporated into the present application by reference. Briefly, the nucleic acid consists of a first region comprising a nucleic acid which encodes the transactivator of the tetracycline-regulated system (tTA) under the control of a moderate promoter and of a second region comprising a nucleic acid of interest which is under the control of a promoter which is sensitive to tTA. This sensitive promoter can be any promoter, even a strong promoter, whose activity is increased in the presence of said transactivator. From a structural point of view, this promoter comprises, in its sequence or at a functional distance from the sequence, at least one site for binding (or operator region Op) the tTA factor. Preferably, the two preceding regions are separate. In order to implement the invention, the NRSE sequences can be placed upstream of the tTA-sensitive promoter, although this is not a prerequisite for activity, including between the two previously described regions.

This particular embodiment of the invention advantageously makes it possible to achieve, at one and the same time, not only regulation of the expression of a nucleic acid by tetracycline but also a tissue specificity for the expression, in particular in nerve cells, which is provided by the NRSE sequences. In addition, this fine regulation of the promoter ensures a remarkable degree of efficacy and reliability in the expression of transgenes, as required in gene therapy.

The present invention also relates to vectors which comprise a nucleic acid such as defined above. While this vector is advantageously able to transduce mammalian, in particular human nerve cells, it is not necessary for it to possess a particular tropism for the said cells. Thus, the invention advantageously makes it possible to use any type of vector. The vector may be of the plasmid (plasmid, episome, artificial chromosome, etc.) or viral type. Of the latter, those which may preferentially be mentioned are vectors derived from adenoviruses, AAVs and herpes viruses, whose tropism for nerve tissues and cells has been thoroughly documented in the prior art. Other viruses such as retroviruses or rhabdoviruses may also be mentioned. In this regard, the examples provided below demonstrate that the activity of NRSE sequences which have been introduced into viral vectors is very substantial. Thus, unexpectedly, when NRSE sequences, in particular 6 and 12 sequences, were introduced into a viral vector (adenovirus), they reduced the expression of a transgene in non-nerve cells by 91 and 98%, respectively, in vitro and by 90 and 96%, respectively, in vivo. These results advantageously demonstrate that it is possible to express a nucleic acid of interest specifically in nerve cells, without expression in non-nerve cells, in accordance with a simple system and due to these regulatory sequences. In addition, these NRSE sequences exhibit the advantage, because of their reduced size, of being particularly well-suited for regulating the expression of genes which are incorporated into a vector in which the space for inserting regulatory sequences is limited. In addition, it is now possible to envisage combining these sequences with other regulatory systems in one and the same vector.

The invention particularly relates, therefore, to a defective recombinant virus which comprises a nucleic acid of interest under the control of expression sequences, characterized in that the said expression sequences comprise a promoter and one or more NRSE sequences.

In general, the recombinant viruses of the invention are defective, that is incapable of replicating autonomously in a cell. The production of defective recombinant viruses is known to the skilled person, and illustrated, for example, in GRAHAM F. and PREVEC L, (In: Methods in Molecular Biology (1991) MURRAY E. J. (ed), The Human Press Inc., Clifton, N.J., chapter 11, pp 109-128). In particular, each of these viruses can be prepared in encapsidation cell lines which possess the said deficient functions. Such cell lines have been described in the literature (293 and derivatives, for example).

According to one particular embodiment of the invention, the defective recombinant virus is an adenovirus. In particular, different serotypes of this virus, the structures and properties of which serotypes vary somewhat, have been characterized. Of these serotypes, preference is given, within the context of the present invention, to using type 2 or type 5 human adenoviruses (Ad 2 or Ad 5) or adenoviruses of animal origin (see application WO94/26914). Adenoviruses of animal origin which can be used within the context of the present invention and which may be mentioned are adenoviruses of canine, bovine, murine (example: Mav1, Beard et al., Virology 75 (1990) 81), ovine, porcine, avian or simian (example: SAV) origin. Preferably, the adenovirus of animal origin is a canine adenovirus, more preferably a CAV2 adenovirus [Manhattan strain or A26/61 (ATCC VR-800), for example]. Preference is given, within the context of the invention, to using adenoviruses of human, canine or mixed origin.

Preferably, the defective adenoviruses of the invention comprise the ITRs, an encapsidation sequence and a nucleic acid according to the invention. Still more preferably, the E1 region at least is non-functional in the genome of the adenoviruses of the invention. The viral gene under consideration can-be rendered non-functional by any technique known to the skilled person, in particular by total deletion, replacement, partial deletion or the addition of one or more bases in the gene(s) under consideration. Such modifications can be obtained in vitro (on the isolated DNA) or in situ, for example using the techniques of genetic engineering, or else by treatment with mutagenic agents. Other regions can also be modified, in particular the E3 (WO95/02697), E2 (WO94/28938), E4 (WO94/28152, WO94/12649, WO95/02697) and L5 (WO95/02697) regions. According to a preferred embodiment, the adenovirus according to the invention contains a deletion in the E1 and E4 regions. According to another preferred embodiment, it comprises a deletion in the E1 region into which are inserted the E4 region and the nucleic acid sequence of the invention (cf. FR94 13355). The recombinant adenovirus can also be a recombinant adenovirus which is defective for the E1 and/or E2 and/or E4 regions, for example. In the viruses of the invention, the deletion in the E1 region preferably extends from nucleotide 455 to nucleotide 3329 in the case of the Ad5 adenovirus sequence.

The defective recombinant adenoviruses according to the invention may be prepared by any technique known to the skilled person (Levrero et al., gene 101 (1991) 195, EP 185 573; Graham, EMBO J. 3 (1984) 2917). In particular, they may be prepared by homologous recombination between an adenovirus and a plasmid which carries, inter alia, a nucleic acid of the invention or a nucleic acid of interest under the control of expression sequences which comprise a promoter and one or more NRSE sequences. The homologous recombination takes place after cotransfection of the said adenovirus and plasmid into an appropriate cell line. The cell line employed should preferably (i) be transformable with the said elements and (ii) contain the sequences which are able to complement the part of the genome of the defective adenovirus, preferably in integrated form in order to avoid the risk of recombination. Examples of cell lines which may be mentioned are the human embryonic kidney cell line 293 (Graham et al., J. Gen. Virol. 36 (1977) 59), which in particular contains, integrated into its genome, the left-hand part of the genome of an Ad5 adenovirus (12%), or cell lines which are able to complement the E1 and E4 functions, such as described, in particular, in applications Nos. WO94/26914 and WO95/02697, or in Yeh et al., J. Virol. 70 (1996) 559. Afterwards, the adenoviruses which have multiplied are recovered and purified using the standard techniques of molecular biology.

According to another particular embodiment of the invention, the defective recombinant virus is an AAV. The adeno-associated viruses (AAV) are DNA viruses of relatively reduced size which integrate stably and site-specifically into the genome of the cells they infect. They are able to infect a wide spectrum of cells without inducing any effect on cell growth, morphology or differentiation. Furthermore, they do not appear to be involved in human pathologies. The AAV genome has been cloned, sequenced and characterized. It comprises approximately 4700 bases and contains, at each end, an inverted repeat region (ITR) of appropriately 145 bases which serves as the origin of replication of the virus. The remainder of the genome is divided into 2 essential regions carrying the encapsidation functions: the left-hand part of the genome, which contains the rep gene, which is involved in viral replication and expression of the viral genes; the right-hand part of the genome, which contains the cap gene encoding the proteins of the virus capsid.

The use of AAV-derived vectors for transferring genes in vitro and in vivo has been described in the literature (see, in particular, WO 91/18088; WO 93/09239; U.S. Pat. No. 4,797,368, U.S. Pat. No. 5,139,941, EP 488 528). These applications describe different AAV-derived constructs in which the rep and/or cap genes are deleted and replaced with a gene of interest, and their use for transferring the said gene of interest in vitro (into cultured cells) or in vivo (directly into an organism). The defective recombinant AAVs according to the invention are defective for all or part of the Rep and/or Cap regions. They can be prepared by cotransfecting a plasmid containing a nucleic acid sequence, or a combination of nucleic acid sequences, of the invention flanked by two AAV inverted repeat regions (ITRs) and a plasmid carrying the AAV encapsidation genes (rep and cap genes) into a cell line which is infected with a human helper virus (for example an adenovirus). An example of a cell line which can be used is the 293 cell line. Other production systems are described, for example, in applications WO95/14771; WO95/13365; WO95/13392 or WO95/06743. The AAV recombinants which are produced are then purified using standard techniques.

According to another particular embodiment of the invention, the defective recombinant virus is a retrovirus, a rhabdovirus or an HSV. The construction of recombinant vectors based on retroviruses and herpes viruses has been widely described in the literature: see, in particular, Breakfield et al., New Biologist 3 (1991) 203; EP 453242, EP 178220, Bernstein et al., Genet. Eng. 7 (1985) 235; McCormick, BioTechnology 3 (1985) 689, etc. In particular, the retroviruses are integrating viruses which selectively infect dividing cells. They therefore constitute vectors of interest for neoplastic applications. The retrovirus genome essentially comprises two LTRs, an encapsidation sequence and three coding regions (gag, pol and env). In the recombinant vectors which are derived from retroviruses, the gag, pol and env genes are generally entirely or partly deleted and replaced with a, heterologous nucleic acid sequence of interest. These vectors can be prepared from various types of retrovirus such as, in particular, MoMuLV (Moloney murine leukaemia virus; also designated MoMLV), MSV (Moloney murine sarcoma virus), HaSV (Harvey sarcoma virus), SNV (spleen necrosis virus), RSV (Rous sarcoma virus) or Friend virus.

In order to construct recombinant retroviruses according to the invention, which retroviruses comprise a nucleic acid of the invention or a nucleic acid of interest under the control of expression sequences comprising a promoter and one or more NRSE sequences, a plasmid which comprises, in particular, the LTRS, the encapsidation sequence and the said nucleic acid sequence is constructed and then used to transfect a so-called encapsidation cell line which is able to supply in trans the retroviral functions which are deficient in the plasmid. In general, the encapsidation cell lines are therefore able to express the gag, pol and env genes. Such encapsidation cell lines have been described in the prior art, in particular the cell line PA317 (U.S. Pat. No. 4,861,719), the cell line PsiCRIP (WO90/02806) and the cell line GP+envAM-12 (WO89/07150). In addition, the recombinant retroviruses can contain alterations within the LTRs for suppressing transcriptional activity and extended encapsidation sequences which include a part of the gag gene (Bender et al., J. Virol 61 (1987) 1639). The recombinant retroviruses which have been produced are then purified using standard techniques.

The present application demonstrates the possibility of using NRSE sequences for regulating the expression of a gene in vivo. Thus, the results which are presented in the examples below demonstrate that the sequences are always active in vivo and enable a transgene to be expressed in neuronal cells without there being any expression in non-neuronal cells.

The application also demonstrates that the NRSE sequences are able, depending on their arrangement (number of copies), efficiently to regulate the activity of a strong promoter, making possible a large number of particularly advantageous uses. Thus, the results obtained demonstrate that the expression in neuronal cells is not only specific but also comparable to that obtained with strong promoters. The invention also demonstrates, astonishingly, that the activity of the NRSE sequences appears to be potentiated when these sequences are inserted into a vector of viral origin. This latter type of construct therefore combines particularly attractive properties such as efficiency of transfer and selectivity of expression.

The invention also relates to any cell which contains a nucleic acid or a vector or a virus such as defined above. Advantageously, this cell is a mammalian nerve cell. It can, in particular, be a cell which derives from an established cell line or a cell which derives from a primary culture. These cells can be used for producing polypeptides, for example, or for testing the activity of genes. They can also be used in cell therapy approaches, by being implanted or injected into a subject.

In this regard, the invention also relates to a composition which comprises a nucleic acid or a vector or a virus or a cell as defined above and an excipient. Advantageously, the composition is a pharmaceutical composition. For their use in accordance with the present invention, the nucleic acid, the vector or the cells are preferably combined with one or more pharmaceutically acceptable excipients in order to be formulated with a view to administration by the topical, in particular stereotactic, oral, parental, intranasal, intravenous, intramuscular, subcutaneous, intraocular, transdermal, etc. route. Preferably, the nucleic acid, the vector or the cells are used in injectable form. This injectable form can, in particular, be sterile, isotonic saline (monosodium or disodium phosphate, sodium, potassium, calcium or magnesium chloride, etc., or mixtures of such salts) solutions, or dry, in particular lyophilized compositions which, by the addition of sterilized water or physiological saline, as the case may be, make it possible to constitute injectable solutions.

According to a preferred mode, the composition according to the invention is administered by the intramuscular route, preferably by means of injection. This is because the chosen intramuscular route makes it possible, unexpectedly, to obtain a substantial therapeutic effect due to the backward transport of the products which are derived from the therapeutic genes and produced in the muscle. Thus, these products, which correspond to the nucleic acids or are derived from the vectors according to the invention, are absorbed at the level of the neuromuscular junctions (motor end-plates) and are forwarded up to the cell bodies of the motor neurones by means of backward transport along the motor neuronal axons. In addition, this mode of administration also has the advantage of avoiding the undesirable effects due to the ectopic expression of therapeutic genes in the treatment of neurological diseases. When this method is used, it becomes readily possible, therefore, to infect the neuronal cells specifically without promoting transgene expression in the injected muscles or excretion of their products into the blood circulation. Thus, this method unquestionably overcomes all the drawbacks which have thus far been encountered in therapy due to the limited access of the neurotrophic factors to the motor neurones, to the very short half-life of these proteins and to the deleterious effects incurred when these products are administered systemically. Furthermore, given the ease of access of the site to be injected, this method can advantageously be used whatever the type of neuronal or motor neuronal pathology.

The quantities of these different elements can be adjusted by the skilled person in accordance with the applications envisaged and in accordance with different parameters such as, in particular, the site of administration under consideration, the number of injections, the gene to be expressed or the duration of the sought-after treatment.

The invention also relates to the use, for preparing a composition which is intended for transferring a nucleic acid of interest and expressing it in a tissue or nerve cell, of a construct which comprises:
a promoter
one or more NRSE sequences, and
the said nucleic acid,
   arranged such that the expression of the said nucleic acid is controlled by the said promoter and such that the activity of the said promoter is controlled by the said sequence(s).

The use of the nucleic acids of the invention for transferring genes in vivo or ex vivo, for example in gene therapy, makes it possible to limit the ectopic expression of the transgenes of interest and to target the sought-after therapeutic effect to the neuronal populations. It thus prevents the deleterious effects which may possibly arise due to diffusion of the transgene into the organism. It is possible to envisage using this system in the various (degenerative or traumatic) pathologies of the spinal cord or any other central, peripheral or neuropsychiatric neurological pathology which specifically affects neuronal populations. In basic neurology, this system should also make it possible to clarify the origin (neuronal or glial) of the effects observed in vitro and in vivo.

The invention also relates to the use, for preparing a composition which is intended for treating motor neuronal diseases, by way of the intramuscular route, with a nucleic acid of interest in a tissue or nerve cell, of a construct which comprises:
a promoter
one or more NRSE sequences, and
the said nucleic acid,
   arranged such that the expression of the said nucleic acid is controlled by the said promoter and such that the activity of the said promoter is controlled by the said sequence(s).

The invention also relates to the use of the vectors, which are as previously defined and which comprise a nucleic acid of interest under the control of expression sequences, characterized in that the said expression sequences comprise a promoter and one or more NRSE sequences.

The use of viral vectors is based on the natural transfection properties of viruses. These vectors prove to be particularly effective as regards transfection. It is thus possible to use the vectors of the invention for transferring a gene of interest and expressing it in neuronal cells, in vivo, in vitro or ex vivo, in particular for the purpose of gene therapy. Thus, expression of a nucleic acid of interest under the control, in particular, of one or more NRSE sequences is obtained specifically in nerve cells, whereas ectopic expression is limited. These vectors may therefore be used in treating and/or preventing various central, peripheral or neuropsychiatric neurological pathologies which affect the nerve cells, in particular neurodegenerative diseases and various pathologies of the spinal cord. Examples of pathologies which may in particular be mentioned are Alzheimer's disease, Parkinson's disease, spinal muscle ataxia and Huntington's chorea.

These vectors can, in particular, be used in treating and/or preventing motor neuronal pathologies such as, for example, amyotrophic lateral sclerosis, type I (Werdnig Hoffmann's disease) and type II or III (Kugelberg-Welander's disease) spinal amyotrophy and spinal bulbar amyotrophy (such as Kennedy's disease).

These vectors are particularly suitable for treating and/or preventing these pathologies by the intramuscular route. As previously explained, this therapeutic route makes it possible to reach the motor neurones as a result of backward transport.

More precisely, the invention relates to the use, for preparing a composition which is intended for transferring a nucleic acid of interest and expressing it in a tissue or nerve cell, of a vector which is as previously defined and which comprises a construct in which:
a promoter,
one or more NRSE sequences, and
the said nucleic acid, are arranged such that the expression of the said nucleic acid is controlled by the said promoter and such that the activity of the said promoter is controlled by the said sequence(s).

The invention also relates to the use, for preparing a composition which is intended for treating motor neuronal diseases, by the intramuscular route, with a nucleic acid of interest in a tissue or nerve cell, of a vector which is as previously defined and which comprises a construct in which:
a promoter,
one or more NRSE sequences, and
the said nucleic acid, are arranged such
   that the expression of the said nucleic acid is controlled by the said promoter and such that the activity of the said promoter is controlled by the said sequence(s).

The invention additionally relates to a method for regulating the expression of genes in vivo, which method comprises inserting NRSE sequences upstream of the said gene and administering the resulting construct and/or the vector comprising the said construct in vivo.

The present application will be described in more detail with the aid of the examples which follow and which should be regarded as being illustrative and not limiting. These examples first of all describe the cloning of an increasing number of NRSE sequences upstream of the PGK promoter in plasmids containing the luciferase reporter gene and selection of constructs which lead to expression of the luciferase being decreased after non-neuronal cells have been transfected. Subsequently, corresponding adenoviral constructs were generated and tested in vitro by infecting cell lines and primary cultures; they were then tested in vivo following intramuscular injection of the recombinant adenoviruses into mice. The results which are presented illustrate the advantages of the nucleic acids of the invention.

### FIGURE LEGENDS

FIG. 1: Diagrammatic depiction of the plasmids which carry a chimeric promoter according to the invention.

FIG. 2: Study of the functional capacity of the plasmids, which was performed by transfecting them into nerve (PC12) (FIG. 2B) and non-nerve (3T3) (FIG. 2A) cells and then measuring the luciferase activity.

FIG. 3: Study of the functional capacity of the defective recombinant viruses in vitro, which was performed by measuring the luciferase activity following infection:

of non-nerve cells (rat 3T3 fibroblasts) (FIG. 3A)

of nerve cells (rat PC12 pheochromocytoma) (FIG. 3B)

of primary non-nerve cell cultures, infection of primary neonate rat kidney cultures (FIG. 3C)

of primary nerve cell cultures, infection of primary neonate rat-derived superior cervical ganglion cultures (FIG. 3D)

of primary non-nerve cell cultures, infection of primary rat embryo-derived astrocyte cultures (FIG. 3E)

of primary nerve cell cultures, infection of primary rat embryo-derived cortical neuronal cell cultures (FIG. 3F)

FIG. 4: Study of the functional capacity of the defective recombinant viruses in vivo, which was performed by injecting them intramuscularly into mice and measuring the luciferase activity.

FIG. 5: Study of the functional capacity of the defective recombinant viruses in vivo, which was performed by injecting them into the tongue muscles of mice and measuring the luciferase activity.

FIG. 5A: Measurement of the luciferase activity in the tongue muscle cells on days 8 and 35.

FIG. 5B: Measurement of the luciferase activity in the nervous system cells (bulbus) on days 8 and 35.

### MATERIALS AND METHODS

Cell Lines and Primary Cultures:

The rat pheochromocytoma (PC12)-derived cells are cultured in RPMI 1640 (Gibco) medium containing 15% foetal calf serum (FCS) (Boehringer). The rat fibroblast-derived 3T3 cells are maintained in Dulbecco's modified Eagle's medium (DMEM) containing 10% foetal calf serum. The primary superior cervical ganglion (SCG) cultures are obtained from dissections of the superior cervical ganglia of 1 or 2-day-old neonate Wistar (Iffa-Credo) rats and dissociated with 3 mg of dispase/ml (Boehringer); they are then deposited on plates which have been previously coated with rat tail-derived collagen. The cells are cultured in 0.5 ml of Leibovitz's medium L-15 (Gibco), which is buffered by bicarbonate and contains several factors including 70 ng of NGF (nerve growth factor)/ml, 5% adult rat serum and 10 μM cytosine arabinofuranoside.

The primary kidney cultures are prepared by dissociating 1 or 2-day-old neonate Wistar (Iffa-Credo) rat kidney tissue with trypsin. The cells are then deposited on plates in 10% FCS to allow the cells to divide until they are infected.

The cortical cultures are derived from E17 Sprague-Dawley rat embryos (Iffa-Credo). The cells are obtained by dissection and mechanical dissociation and are cultured in DMEM medium containing 100 μg of transferrin/ml, 25 μg of insulin/ml, 10 μg of putrescine/ml, 5 ng of sodium selenite/ml, 6.3 ng of progesterone/ml and 2 mM glutamine (Sigma).

The primary astrocyte cultures are derived from rat embryonic cortical tissue (E17, Sprague-Dawley) and cultured in DMEM medium containing 10% FCS in a 10% CO₂/90% air atmosphere.

In Vitro Experiments:

For the transient transfection experiments, one million cells are electroporated using a Bio-Rad system, at 960 μF and 190 V, in the case of the PC12 cells, or 250 V in the case of the 3T3 cells, with 6 μg of each of the plasmids tested, 7 μg of Bluescript plasmid (Stratagene) and 2 μg of pCAT 3 control vector (Promega), which is a plasmid which encodes chloramphenicol acetyltransferase (CAT), which enables the transfection efficiency to be monitored.

The cell cultures are infected by replacing the culture medium with medium which is free of serum and which contains the viral suspension at an MOI of approximately 200 pfu, and incubating for 45 minutes. In the case of each construct (plasmid or adenoviral), 3 luciferase activity values are determined from 3 different wells. For each viral construct, the infection experiments are repeated twice using different stocks of virus.

48 hours after electroporation or infection, the cells are harvested in 200 μl of a lysis buffer containing 25 mM Tris/phosphate, pH 7.8; 0.08 mM luciferin; 0.1 mM ATP; 8 mM MgCl₂; 1 mM dithiothreitol; 1 mM EDTA; 15% glycerol and 1% Triton. The luciferase activity is measured using a LUMAT LB9501 luminometer (Berthold). In the case of the transfected cells, 1% bovine serum albumin (BSA) is added to the lysis buffer and the activity is standardized on the basis of the CAT activity, which is determined by the liquid scintillation counting method. In the case of the recombinant adenoviruses, the luciferase activity is standardized in terms of the protein concentration of the cell extracts, which was determined by the Bio-Rad system (Bio-Rad laboratories).

In-Vivo Experiments:

Six-month-old male C57B16 mice (Charles River) are anaesthetized with a Rompun (Bayer)/Ketamine (UVA) mixture, and the Ad-PGK-luc and Ad-NRSE-PGK-luc constructs are slowly injected (2.5 μl/ml) into the tongue (10⁹ pfu/4×2.5 μl) and/or into the gastrocnemius muscle at a dose of 2.10⁹ pfu/muscle (30 μl). The mice are sacrificed with pentobarbital (Sanofi) at from 7 to 35 days after infection. The bulbi, the tongues and the muscles are removed for measuring the luciferase activity. The bulbi are dissociated mechanically in 200 μl of lysis buffer. The muscles and tongues are dissociated with a DIAX 900 polytron system (Heidolph) in 1 and 2 ml of lysis buffer, respectively.

### EXAMPLES

1. Construction of Chimeric Promoters and Plasmid Vectors

The aim of this example is to describe the preparation of chimeric promoters and the construction of plasmid vectors which contain these particular promoters.

The plasmid pPGK-Luc comprises the Luc gene under the control of the ubiquitous eukaryotic PGK promoter. In order to obtain plasmid pPGK-Luc, the 500 bp of the murine PGK promoter were inserted into a commercial plasmid, pUT 103 (CAYLA FRANCE), which contains the luciferase reporter gene fused to zeocin and an SV 40 polyadenylation sequence in the early configuration (PolyA).

Next, one, two, three, six and twelve copies of the NRSE sequence from the SCG10 gene (TTCAGCACCACGGAGAGTGCC, SEQ ID No. 1) [2] were inserted in an antiparallel configuration upstream of the PGK promoter. For this, a 26 bp fragment containing the above-described NRSE sequence flanked by 2 MluI sites was constructed by hybridizing 2 corresponding synthetic oligonucleotides. This fragment was inserted, in one or more copies, into the MluI site of pPGK-Luc and resulted in plasmids containing 1, 2 and 3 NRSE copies. The plasmid containing 6 NRSE copies was obtained from a plasmid comprising 3 copies (pNRSE3-PGK-Luc) using the following strategy: a BamHI/XhoI fragment containing 3 NRSE copies is removed from pNRSE3-PGK-Luc and introduced into the XhoI site of pNRSE3-PGK-Luc, thereby generating p6NRSE-PGK-Luc. Similarly, the plasmid comprising 12 NRSE copies was obtained from the plasmid comprising 6 copies (p6NRSE-PGK-Luc) by introducing two copies of the BamHI/XhoI fragment into p6NRSE-PGK-Luc.

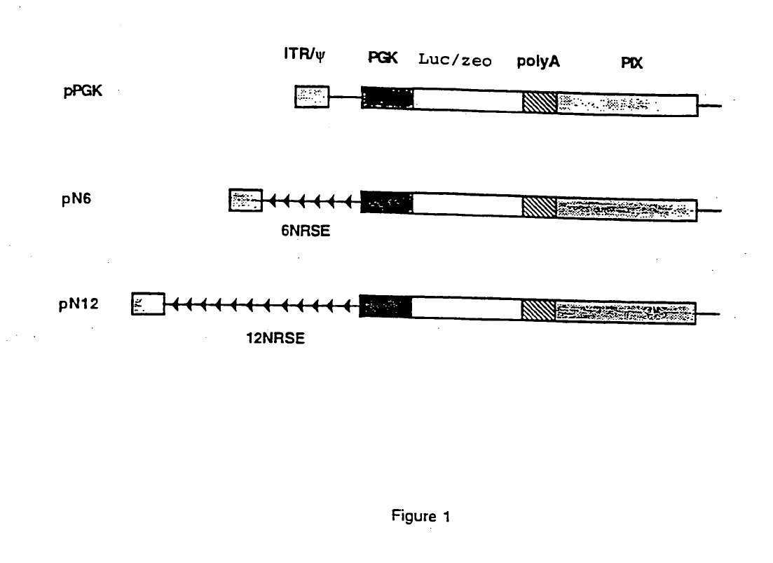

Finally, the PGK (with or without NRSE)-Luc-PolyA expression cassette from the different plasmids described above was introduced between the left-hand adenovirus ITR sequence (inverted terminal repeat, origin of replication) and the sequence encoding the IX polypeptide (viral capsid protein) of a shuttle plasmid which could be used for constructing defective recombinant viruses (FIG. 1).

It is to be understood that the same protocol can be used to construct any other plasmid which incorporates from 1 to 50 copies of an NRSE motif combined with a ubiquitous eukaryotic promoter other than PGK under whose control any transgene of interest can be inserted.

2. Functional Analysis Effected by Transfecting Cell Lines

The aim of this example is to demonstrate that the plasmid constructs which comprise the chimeric promoter containing the NRSE sequences are functional and allow the cellular expression of a gene of interest.

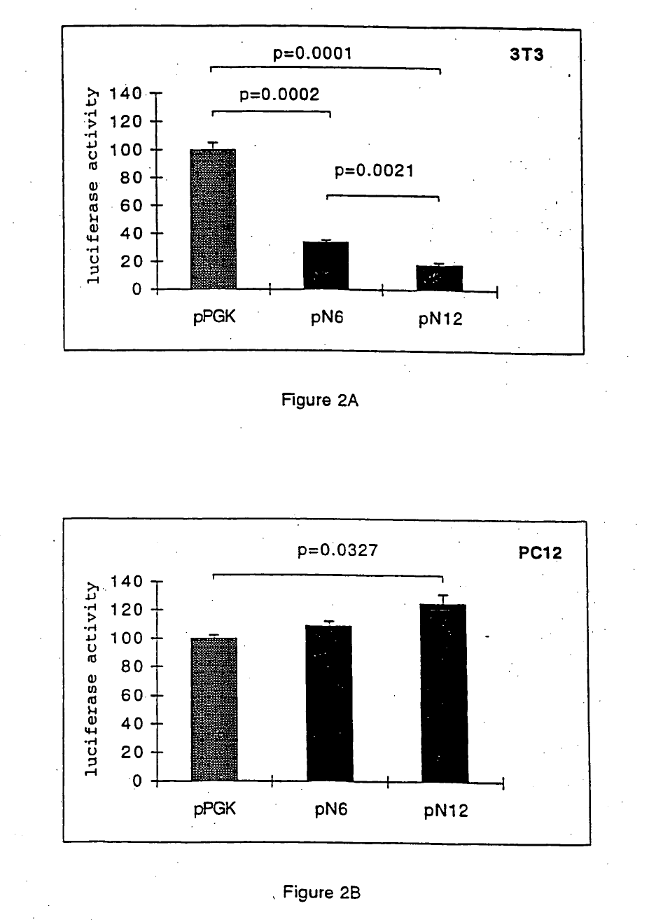

The plasmid constructs described in Example 1 were tested by transiently transfecting (electroporation) PC12 (rat pheochromocytoma) neuronal cell lines and 3T3 (rat fibroblast) non-neuronal cell lines. The resulting luciferase activity values are standardized in relation to the CAT (chloramphenicol acetyltransferase) activity, which is measured in a solution containing 125 mM tris-phosphate, pH 7.8, 125 μM chloramphenicol and 0.12 μM radiolabelled acetyl coenzyme A. The assays are carried out, after incubating at 37° C. for one hour, in the presence of Econofluor scintillation fluid and using a KONTRON counter. Three luciferase activity values were determined for each plasmid construct, and the means and standard errors obtained are presented in FIG. 2.

The results obtained demonstrate that the constructs comprising 6 and 12 NRSE copies are able to decrease expression of the luciferase by 66 and 82%, respectively, in the non-nerve cells (3T3), that is a loss of almost a logarithm of expression as compared with the pPGK-Luc construct. The results also demonstrate that the presence of the NRSE motifs in the PC12 nerve cells does not decrease expression of the luciferase and may indeed even be able to increase it. Thus, a statistically significant increase of 25% is observed between p12NRSE-PGK-Luc and pPGK-Luc.

The results obtained thus demonstrate that the plasmid constructs which contain the chimeric promoter comprising the NRSE sequences are functional and permit cellular expression of a gene of interest solely in the neuronal cells while at the same time very significantly inhibiting expression of the said gene in the non-neuronal cells.

3. Construction of Defective Recombinant Viruses

The aim of this example is to describe the preparation of the chimeric promoters and the construction of viral vectors which contain these particular promoters.

The three corresponding adenoviral constructs were generated with the aim of complementing this study in vitro and in vivo: Ad-PGK-Luc, Ad-6NRSE-PCK-Luc and AD-12NRSE-PGK-Luc. Known biological techniques were used to construct these recombinant viruses by means of cotransfecting the shuttle plasmids described in Example 1 and a defective type 5 adenovirus genome into an encapsidation line (293 cells). The shuttle plasmids were firstly linearized by enzymic cleavage at the FspI site and then cotransfected, together with the long adenovirus ClaI fragment, into cell line 293 using the method of precipitating the DNA with calcium phosphate.

The encapsidated recombinant genomes are then purified using standard techniques, in particular using the plaque-purification technique. The integrated fragment is then verified by restriction fragment analysis and PCR. The virus stocks were prepared by propagating the recombinant adenovirus in 293 cells and then ultracentrifuging, in particular in a caesium chloride gradient, and purifying on a Sephadex G25M (Pharmacia) purification column. The viral titres were determined by optical density reading and were additionally checked by the plaque method. All the virus stocks had titres of about 2.10⁸ pfu/μl.

4. In-Vitro Functional Analysis

The aim of this example is to demonstrate that the viral constructs which are described in Example 3, and which contain the chimeric promoter comprising the NRSE sequences, are functional and enable the promoter to be controlled in different cell types.

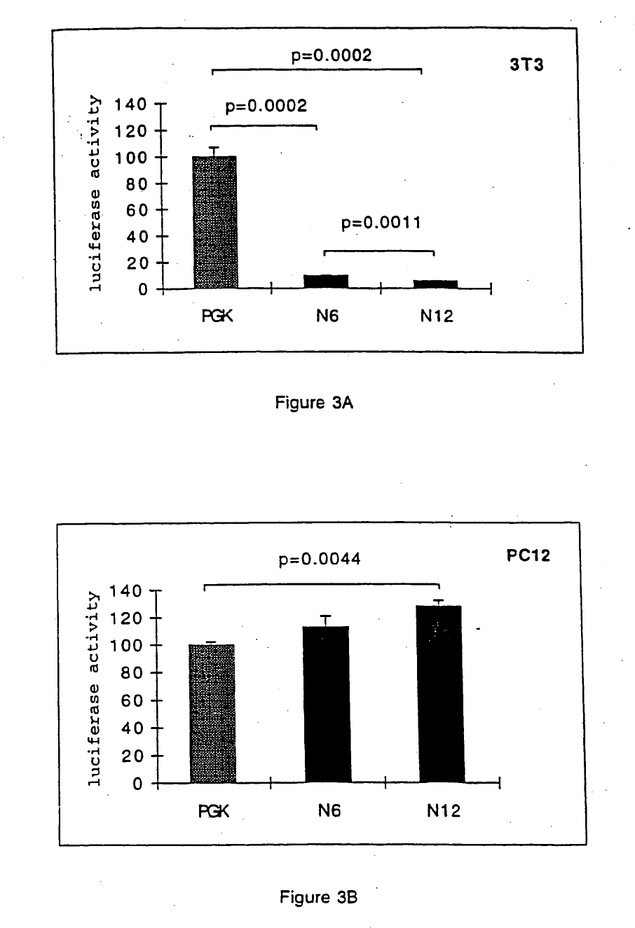

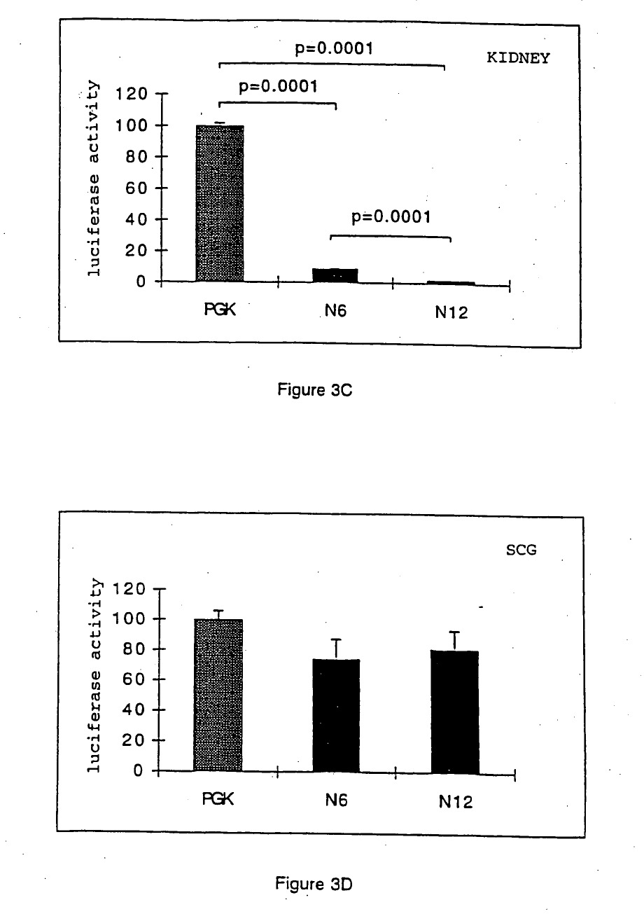

The three adenoviruses which were constructed in Example 3 above were tested in vitro by infecting cell lines and primary cultures with doses of 20 and 200 pfu/cell, respectively. The measurements are carried out on 50 μl of supernatant, as previously described, and standardized in relation to the total protein quantity, obtained using a Bio Rad protein assay kit system (Bio-Rad). For each adenovirus, 3 luciferase activity values were determined in different wells. The mean values and the standard errors obtained are shown in FIG. 3, in the case of the cell lines, and in FIG. 4, in the case of the primary cultures.

When the recombinant adenoviruses Ad-6NRSE-PGK-Luc and Ad-12NRSE-PGK-Luc are used, expression of the luciferase reporter gene is decreased by 97% and 99%, respectively, in the 3T3 non-neuronal cell line.

In the PC12 neuronal cell line, the luciferase activity of the two adenoviruses containing NRSE sequences is higher than that observed when the Ad-PGK-Luc adenovirus is used. In the same way, the luciferase activity obtained with the 3 adenoviral constructs is similar following infection of primary neonate rat superior cervical ganglion (SCG) cultures (a one-factor ANOVA global analysis of variance does not show any significant difference between the three groups). On the other hand, expression of the luciferase is decreased by from 91 to 98% following infection of primary neonate rat kidney cultures with the Ad-6NRSE-PGK-Luc and Ad-12NRSE-PGK-Luc recombinant adenoviruses, that is a loss of 2 logarithms of PGK expression in the presence of 12 NRSE copies.

Confirming the preceding results, infection, by the three constructs, of astrocytes and cells of the neuronal cortex derived from 17-day-old rat embryos shows, as expected, that the luciferase activity is very significantly decreased (by a factor of 10) in the astrocyte cells in the case of the constructs containing 6 or 12 NRSE sequences (FIG. 3E). As is also expected, the luciferase activity of the three types of previously mentioned viral construct is comparable in the cortex cells (FIG. 3F).

These results are particularly surprising and remarkable insofar as they demonstrate (i) that the chimeric promoters of the invention are functional on primary cultures, (ii) that the promoters are functional in a viral context, and (iii) that the repressor activity is greater in the adenoviral context than in the corresponding plasmid vector (cf. FIGS. 2 and 3).

Thus, this example demonstrated that the viral constructs which contain the chimeric promoter comprising the NRSE sequences are functional and permit cellular expression of a gene of interest solely in neuronal cells while very significantly inhibiting expression of the said gene in non-neuronal cells.

5. In-Vivo Functional Analysis

The aim of this example is to demonstrate that the viral constructs which are described in Example 3, and which contain the chimeric promoter comprising the NRSE sequences, are functional and permit control of the promoter in vivo.

In view of the very encouraging results described in Example 4, the activity of the constructs of the invention was tested in vivo by measuring expression of the luciferase following intramuscular injection (gastrocnemius muscle and tongue muscle) of these 3 recombinant adenoviruses into mice.

5.1 Injection into the Gastrocnemius Muscle

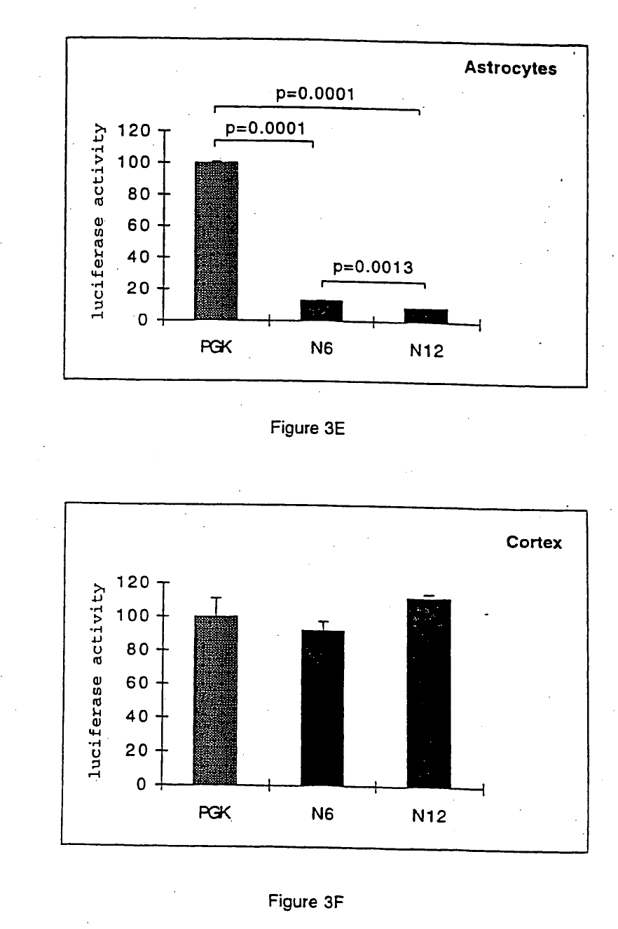

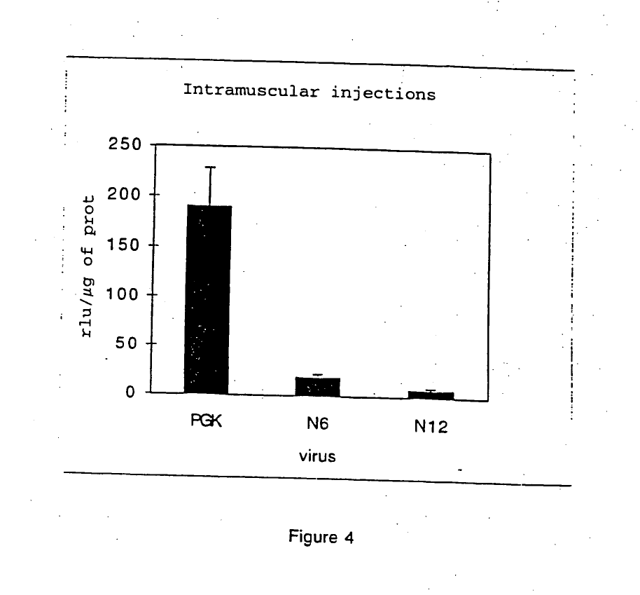

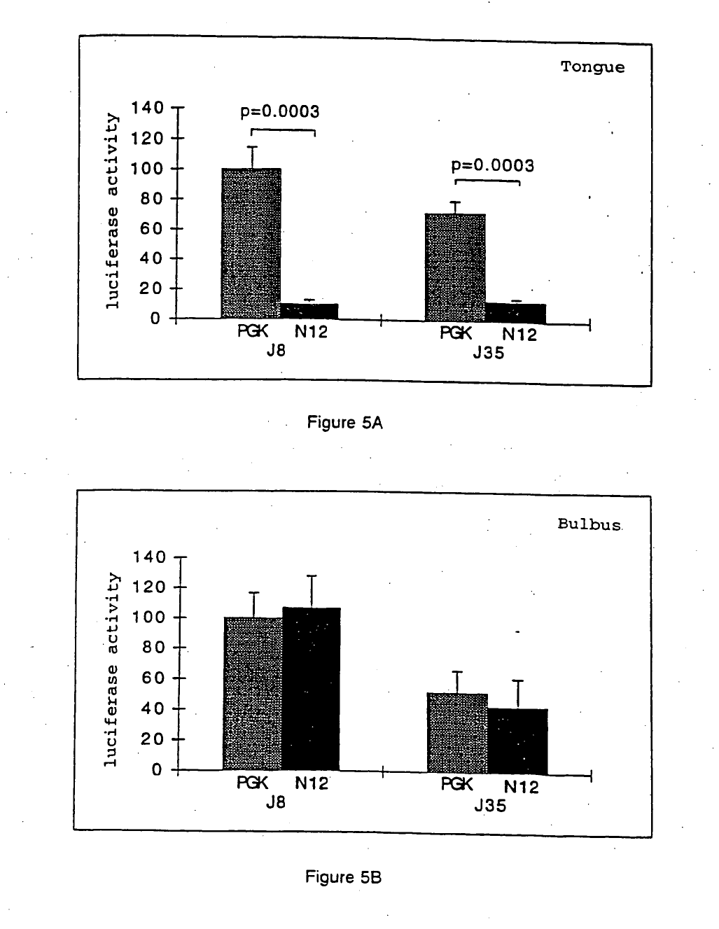

Each adenoviral construct was injected in 3 stages into the right gastrocnemius muscle of 7 mice at a dose of 2.10⁹ pfu/muscle and in a volume of 30 μl. The injected muscles were removed 7 days after the injection and dissociated in 1 ml of buffer with a view to determining the luciferase activity, as carried out on 150 μl of supernatant. The means and standard errors of the 7 luciferase activity values obtained, related to the quantity of protein, are given in FIG. 5.

These results show that, when the Ad-6NRSE-PGK-Luc and Ad-12NRSE-PGK-Luc recombinant adenoviruses are used, expression of the luciferase is decreased by 90 and 96% one week after the injections.

The luciferase expression following the intramuscular injection of Ad-12NRSE-PGK-Luc and Ad-PGK-Luc was compared in mice over a longer term. When Ad-12NRSE-PGK-Luc is used, the luciferase activity is always found to be decreased one month (95%) and three months (91%) after the injections.

5.2 Injection into the Tongue Muscles

The backward transport was used in order to compare expression of the two constructs Ad-PGK-Luc and Ad-12NRSE-PGK-Luc in the bulbus and in the tongue muscles following intramuscular injection into the mouse tongue.

In a comparable manner to the results already obtained, the luciferase activities of the two constructs in the nervous system are similar at one week after administration (FIG. 5B). Conversely, the luciferase activity decreases by 90% in the muscles injected with the Ad-12NRSE-PGK-Luc construct as compared with the muscles injected with the Ad-PGK-Luc construct (FIG. 5A). This repression is maintained over time and for at least 35 days after infection. Interestingly, even though the level of expression at 35 days is lower than that obtained at 8 days after the infection, the difference in expression between the two previous viral constructs remains very significant.

Once again, these results confirm that the constructs of the invention function in vivo, and demonstrate a repression factor which is even greater than those observed in vitro following transfection of the plasmids into the non-neuronal cells.

These results additionally demonstrate the high degree of efficiency of the constructs of the invention in vivo for targeting expression of a gene of interest into the neuronal cells. The use of this approach in gene therapy makes it possible to limit the ectopic expression of the transgenes of interest and prevents the deleterious effects which may possibly arise due to diffusion of the transgene into the orgnism.

### LITERATURE

[1] Mori N., Stein R., Sigmund O. and Anderson D. J. Neurone, 4: 583-594 (1990)
[2] Mori N., Schoenherr C., Vandenbergh D. J. and Anderson D. J. Neurone, 9: 45-54 (1992)
[3] Li L., Suzuki T., Mori N. and Greencard P. PNAS USA, 90: 1460-1464 (1993)
[4] Schoenherr C. J., Paquette A. J. and Anderson D. J. PNAS USA, 93: 9981-9886 (1996)
[5] Schoenherr C. J. and Anderson D. J. Science, 267: 1360-1363 (1995)
[6] McBurney, Sutherland, Adra, Leclair, Rudnicki, Jardine Nucleic Acids Research, Vol. 19, No. 20: 5755-5761 (1991)
[7] Moullier P., Marechal V., Danos O. and Heard J. M. Transplantation, 56: 427-432 (1993)
[8] McDonald R. J., Lukason M. J., Raabe O. G., Canfield D. R., Burr E. A., Kaplan J. M., Wadsworth S. C. and St George J. A. Hum. Gene Ther., 8: 411-422 (1997)
[9] Maue R. A., Kraner S. D. Goodman R. H. and Mandel G. Neurone, 4: 223-231 (1990)
[10] Bessis A., Champtiaux N., Chatelin L. and Changeux J. P. PNAS USA, 94: 5906-5911 (1997).

1-37. (canceled)

38. A method of neuronal-specific expression of a nucleic acid of interest in neuronal cells comprising the steps of:
a. introducing the nucleic acid of interest into the neuronal cells; and
b. growing the cells under conditions allowing expression of the nucleic acid of interest.

39. The method according to claim 38, wherein the nucleic acid of interest comprises the nucleic acid of interest operably linked to a chimeric promoter, which comprises:
a strong ubiquitous promoter from a first gene, and
at least one neuron restrictive silencer element (NRSE) sequence from a second gene,
wherein the strong ubiquitous promoter is operably linked to the at least one NRSE sequence, and wherein the strong ubiquitous promoter has promoter activity comparable to the CMV (cytomegalovirus), RSV (Rous sarcoma virus), SV40 (simian virus 40), or LTR (long terminal repeat) promoters.

40. The method according to claim 39, wherein the strong ubiquitous promoter is a eukaryotic promoter or a viral promoter which is active in nerve cells or nerve tissue.

41. The method of claim 39, wherein the strong ubiquitous promoter is a eukaryotic promoter.

42. The method according to claim 40, wherein the strong ubiquitous promoter is a house-keeping gene promoter.

43. The method according to claim 42, wherein the strong ubiquitous promoter is selected from the promoters of the phosphoglycerate kinase (PGK), Ef1α, β-actin, vimentin, adolase A or α1-antitrypsin genes.

44. The method according to claim 39, wherein the chimeric promoter comprises from 1 to 20 NRSE sequences.

45. The method according to claim 44, wherein the chimeric promoter comprises 3, 6 or 12 NRSE sequences.

46. The method according to claim 39, wherein the at least one NRSE sequence comprises all or part of the sequence SEQ ID No. 3, wherein N is A, G, C or T.

47. The method of claim 46, wherein the at least one NRSE sequence is selected from all or part of the sequences SEQ ID Nos. 1, 2 or 4-11.

48. The method of claim 39, wherein the NRSE sequence or sequences are placed upstream of the strong ubiquitous promoter.

49. The method of claim 39, wherin the chimeric promoter comprises regulatory elements in addition to the NRSE sequences.

50. The method of claim 49, wherein the regulatory element is a tetracycline operator/repressor system.

51. A method of neuronal-specific expression of a nucleic acid of interest in neuronal cells from a viral vector, comprising the steps of:
a. introducing the nucleic acid of interest into the neuronal cells; and
b. growing the cells under conditions allowing expression of the nucleic acid of interest.

52. The method according to claim 51, wherein the nucleic acid of interest comprises the nucleic acid of interest operably linked to a chimeric promoter, which comprises:
a promoter, and
at least one neuron restrictive silencer element (NRSE) sequence, wherein the promoter is operably linked to the at least one NRSE sequence.

53. A method of expressing a replication defective recombinant virus selectively in neuronal cells comprising the steps of:
a. expressing the nucleic acid of interest under the control of expression sequences, wherein said expression sequences comprise a promoter and one or more NRSE sequences; and
b. growing the cells under conditions allowing expression of the nucleic acid of interest.

54. The method according to claim 53, wherein the virus is an adenovirus.

55. The method according to claim 53, wherein the virus is an Adeno Associated Virus (AAV).

56. The method according to claim 53, wherein the virus is a retrovirus.

57. The method according to claim 53, wherein the virus is a rhabdovirus.

58. The method according to claim 38, wherein the neuronal-specific expression of the nucleic acid of interest is within an isolated cell.

59. The method according to claim 58, wherein the cell is a mammalian nerve cell.

60. The method according to claim 38, wherein the neuronal-specific expression of the nucleic acid of interest is expressed from a composition comprising the chimeric promoter and a physiologically acceptable excipient.

61. A method of neuronal-specific expression of a nucleic acid of interest in neuronal cells, wherein the nucleic acid of interest is operably linked to a chimeric promoter, comprising the steps of:
a. introducing the nucleic acid of interest into the neuronal cells; and
b. growing the cells under conditions allowing expression of the nucleic acid of interest.

62. The method according to claim 61, wherein the chimeric promoter is xNRSE-PGK, which comprises a PGK promoter and xNRSE sequences, wherein x is an integer from 1 to 50.

63. The method according to claim 39, wherein the chimeric promoter comprises from 3 to 15 NRSE sequences.

64. The method according to claim 51, wherein the neuronal-specific expression of the nucleic acid of interest is expressed from a composition comprising the viral vector and a physiologically acceptable excipient.

65. The method according to claim 58, wherein the cell is an ex vivo cell.

66. The method according to claim 59, wherein the cell is an ex vivo cell.

67. The method according to claim 39, wherein the chimeric promoter comprises at least two NRSE sequences, wherein each of the at least two NRSE sequences comprise all or part of SEQ ID No. 3, and wherein N is A, G, C or T.

68. The method according to claim 39, wherein the chimeric promoter comprises at least two NRSE sequences, wherein each of the at least two NRSE sequences comprise sequences SEQ ID Nos. 1, 2 or 4-11, or functional variants of these sequences.
